(19) United States
(12) Patent Application Publication
(10) Pub. No.: US 2008/0206799 A1
(43) Pub. Date: Aug. 28, 2008

(54) CARBOHYDRATE RATIO TESTING USING FREQUENT BLOOD GLUCOSE INPUT

(76) Inventor: Michael Blomquist, Blaine, MN (US)

Correspondence Address: SCHWEGMAN, LUNDBERG & WOESSNER, P.A., P.O. BOX 2938, MINNEAPOLIS, MN, 55402, US

(21) Appl. No.: 11/679,712

(22) Filed: Feb. 27, 2007

(51) Int. Cl. A61B 5/145; C12Q 1/54

(52) U.S. Cl. 435/14; 600/365

(57) ABSTRACT

An apparatus comprising a user interface configured to generate an electrical signal to begin a carbohydrate ratio test when prompted by a user, an input configured to receive sampled blood glucose data of a patient that is obtained during a specified time duration (including a time duration after delivery of an initial carbohydrate insulin bolus), and a controller in electrical communication with the input and the user interface. The controller includes a carbohydrate ratio module configured to establish a blood glucose baseline from a measure of an initial blood glucose level of the patient, and determine a carbohydrate ratio according to a difference between a blood glucose level of the patient at the end of the specified time duration and the blood glucose baseline. Other systems and methods are disclosed.

### TECHNICAL FIELD

The field generally relates to patient insulin management devices and, in particular, but not by way of limitation, to systems, devices, and methods for adjusting insulin therapy.

### BACKGROUND

People who suffer from diabetes require insulin to keep their blood glucose level as close as possible to normal levels. It is essential for people with diabetes to manage their blood glucose level to within a normal range. Complications from diabetes can include heart disease (cardiovascular disease), blindness (retinopathy), nerve damage (neuropathy), and kidney damage (nephropathy). Insulin is a hormone that reduces the level of blood glucose in the body. Normally, insulin is produced by beta cells in the pancreas. In non-diabetic people, the beta cells release insulin to satisfy two types of insulin needs. The first type is a low-level of background insulin that is released throughout the day. The second type is a quick release of a higher-level of insulin in response to eating. Insulin therapy replaces or supplements insulin produced by the pancreas.

Conventional insulin therapy typically involves one or two injections a day. The low number of injections has the disadvantage of allowing larger variations in a person's insulin levels. Some people with diabetes manage their blood glucose level with multiple daily injections (MDI). MDI may involve more than three injections a day and four or more blood glucose tests a day. MDI offers better control than conventional therapy. However, insulin injections are inconvenient and require a diabetic person to track the insulin doses, the amount of carbohydrates eaten, and their blood glucose levels among other information critical to control.

Blood glucose (BG) management devices help a diabetic person manage their blood glucose. For example, an insulin pump is a BG management device that provides insulin throughout the day. A glucose monitor (GM) or glucose meter is a BG management device to measure blood glucose levels. Some monitors require a finger-stick to acquire a sample of blood that is applied to a test strip to get a blood glucose reading. Some monitors are able to provide continuous monitoring of blood glucose. Other BG management devices include computers running software to help a diabetic person manage insulin therapy. However, most BG management devices are limited in the control over blood glucose that they offer.

### SUMMARY

This document discusses, among other things, apparatuses and methods for managing insulin therapy. An apparatus example includes a user interface configured to generate an electrical signal to begin a carbohydrate ratio test when prompted by a user, an input configured to receive sampled blood glucose data of a patient that is obtained during a specified time duration (including a time duration after delivery of an initial carbohydrate insulin bolus), and a controller in electrical communication with the input and the user interface. The controller includes a carbohydrate ratio module configured to establish a blood glucose baseline from a measure of an initial blood glucose level of the patient, and to determine a carbohydrate ratio according to a difference between a blood glucose level of the patient at the end of the specified time duration and the blood glucose baseline.

A method example includes receiving a user prompt in a blood glucose (BG) management device to start a carbohydrate ratio test, receiving sampled blood glucose data of a patient obtained during a specified time duration (including a time duration after delivery of an initial carbohydrate insulin bolus), establishing a blood glucose baseline from at least one measure of a blood glucose level of the patient, and determining a carbohydrate ratio, using the BG management device, according to a difference between the blood glucose baseline and the blood glucose level of the patient after the specified time duration.

This summary is intended to provide an overview of the subject matter of the present patent application. It is not intended to provide an exclusive or exhaustive explanation of the invention. The detailed description is included to provide further information about the subject matter of the present patent application.

### BRIEF DESCRIPTION OF THE DRAWINGS

FIG. 1 is a block diagram of portions of a blood glucose (BG) management device.

FIG. 2 is an example illustration of a graph of blood glucose during a carbohydrate ratio test.

FIG. 3 is a block diagram of portions of an example of a BG management device that includes a pump mechanism.

FIG. 4 is an illustration of a BG management device that includes an insulin pump.

FIG. 5 is another block diagram of portions of a BG management device that includes a pump mechanism.

FIG. 6 is a block diagram of a BG management device that includes a blood glucose sensor circuit.

FIG. 7 is a block diagram of portions of another example of a BG management device.

FIG. 8 is a flow diagram of a method of automatically determining a carbohydrate ratio using blood glucose data.

### DETAILED DESCRIPTION

In the following detailed description, reference is made to the accompanying drawings which form a part hereof, and specific embodiments in which the invention may be practiced are shown by way of illustration. It is to be understood that other embodiments may be used and structural or logical changes may be made without departing from the scope of the present invention.

It is important for a diabetic person to be treated with the proper amount of insulin. As discussed previously, high blood sugar can lead to serious complications. Conversely, a person with low blood sugar can develop hypoglycemia. Ideally, insulin therapy mimics the way the body works. An insulin pump is one way to mimic the body's insulin production. An insulin pump can provide a background or basal infusion of insulin throughout the day and provide a quick release or bolus of insulin when carbohydrates are eaten. If a person develops high blood sugar, a correction bolus can be delivered by the pump to correct it. While insulin pumps improve convenience and flexibility for a diabetic person, they can be sophisticated devices. Some insulin pumps can be difficult to program. Proper use of an insulin pump requires a user to go through a learning curve to properly use and program the pump.

A carbohydrate ratio refers to the amount of carbohydrates covered by a unit of insulin. It is sometimes referred to as a carbohydrate factor, or carb factor, and is typically specified as grams of carbohydrates per unit of insulin. An insulin pump may use the carbohydrate ratio to automatically determine a carbohydrate insulin bolus amount required to match a number of carbohydrates ingested by the patient, or at least to keep post-meal blood glucose within a range that is healthy for a patient. For example, the patient may plan to eat seventy grams of carbohydrates. If the carbohydrate ratio is ten grams of carbohydrates per unit of insulin, the insulin pump would determine that seven units of insulin are required to cover the carbohydrates.

The appropriate carbohydrate ratio may vary from person to person, yet it is important for a pump to use an appropriate carbohydrate ratio. If a carbohydrate ratio is too small, the pump may determine a carbohydrate bolus that is too large for the carbohydrates consumed. This may cause a low blood glucose level within a few hours of the carbohydrate bolus (e.g., the blood glucose level drops below 70 mg/dl). If a carbohydrate bolus is too large, the pump may determine a carbohydrate bolus that is too small for the carbohydrates consumed. This may cause a high blood glucose level within a few hours of the carbohydrate bolus.

Typically, the carbohydrate ratio for a pump is initially entered by a clinician based on a total daily dose (TDD) of insulin for the diabetic person. The clinician may use a formula such as the “500 rule” in setting the carbohydrate ratio. For example, if a person's TDD is 40 units of insulin, the carbohydrate ratio would be 500/40 or about 13 grams per unit of insulin. The clinician may also take into account factors such as a person's age, weight, and activity level when setting the carbohydrate ratio. Other formulas include the 550 rule and the 600 rule. For example, under the 600 rule the carbohydrate ratio would be 600/40 or 15 grams per unit of insulin. As discussed above, the larger the carbohydrate ratio, the smaller a carbohydrate bolus becomes. A clinician may prefer one rule over another based on experience; including rules that are not based on TDD.

Once an approximate carbohydrate ratio has been established using TDD or some other method, the patient's actual or most effective carbohydrate ratio should be determined. However, determining such a carbohydrate ratio is complicated by the fact that an appropriate carbohydrate ratio varies from person to person, may be different for a person at various times of the day, and may change for a person over time. A diligent insulin pump user may adjust their carbohydrate ratio many times as they try to find their appropriate carbohydrate ratio and determine how it may vary with time and how it may vary under other circumstances. Blood glucose (BG) management devices are more valuable to a diabetic person if the device conveniently assists them in determining their appropriate carbohydrate ratio.

### Apparatus Embodiments

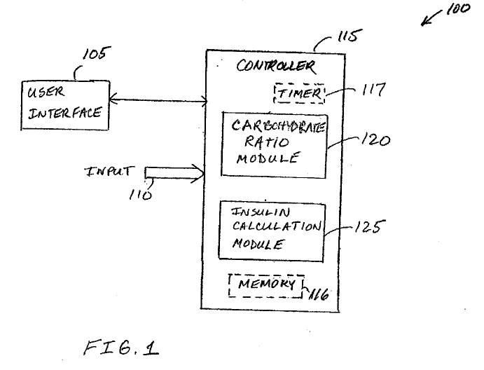

FIG. 1 is a block diagram of portions of a BG management device 100. Examples of a BG management device 100 include, among other devices, an insulin pump, a blood glucose monitor (GM) or meter, and a computing device running software to assist a diabetic patient in managing insulin therapy. The BG management device 100 includes a user interface 105, an input 110, and a controller 115 in electrical communication with the input 110 and the user interface 105. The user interface 105 generates an electrical signal received by the controller 115 to begin a carbohydrate ratio test when prompted by a user. The user interface may include a pushbutton, keypad, or a computer mouse. The user interface may include a display to provide instructions to the user. The display may include a touch-screen. The user of the device may be a clinician, other caregiver, or a diabetic patient. The user prompts the BG management device 100 using the user interface 105 to begin a carbohydrate ratio test.

The controller 115 can be implemented using hardware circuits, firmware, software or any combination of hardware, firmware and software. Examples, include a microcontroller, a logical state machine, and a processor such as a microprocessor, application specific integrated circuit (ASIC), or other type of processor. The controller 115 includes a carbohydrate ratio module 120. Modules can be software, hardware, firmware or any combination of software, hardware, and firmware. Multiple functions can be performed in one or more modules.

The carbohydrate ratio module 120 determines a carbohydrate ratio. In some embodiments, an initial carbohydrate ratio is calculated using a formula such as the 500 rule and the initial carbohydrate ratio is manually entered by a user through the user interface. In some embodiments, the BG management device calculates the initial carbohydrate ratio. For example, the carbohydrate ratio module 120 may be configured for receiving daily injection information (e.g., MDI information) entered by a user through the user interface 105. The daily injection information provides a measure of TDD. The carbohydrate ratio module 120 estimates the initial carbohydrate ratio using the daily injection information. For example, a clinician may prefer to program the carbohydrate ratio module 120 to use a calculation such as the 500 rule. The carbohydrate ratio module 120 then an initial carbohydrate ratio module 120 using the TDD and the 500 rule. In other examples, the carbohydrate ratio module 120 may use a rule desired by a clinician that is different from the 500 rule, 550 rule, or 600 rule.

The carbohydrate ratio module 120 establishes a blood glucose baseline using one or measures of an initial blood glucose level of the patient. In some examples, the carbohydrate ratio module 120 aggregates multiple measures of a patient's blood glucose level into a baseline measurement. The carbohydrate ratio module 120 may use an average of the multiple measurements to establish such a baseline. In some embodiments, the carbohydrate ratio module 120 establishes multiple blood glucose baselines associated with different times of the day to account for any daytime variation of the patient's blood glucose level.

As part of the carbohydrate ratio test, the patient receives an initial carbohydrate insulin bolus. A carbohydrate insulin bolus is an amount of insulin delivered to match or cover carbohydrates in an upcoming meal. If the BG management device 100 includes an insulin pump, the carbohydrate insulin bolus may be delivered using the BG management device 100. If the BG management device 100 does not include an insulin pump, the carbohydrate insulin bolus may be delivered using a separate device that includes an insulin pump or may be delivered by injection. In some embodiments, the BG management device includes an insulin calculation module 125 to calculate the amount of insulin in the initial bolus based on the initial carbohydrate ratio and the amount of carbohydrates ingested by the patient. If the BG management device 100 includes a pump, the BG management device may display the amount of insulin in the carbohydrate insulin bolus and wait for confirmation from the patient before delivering the carbohydrate insulin bolus. If the BG management device 100 doe not include a pump, the BG management device 100 may instruct the patient to deliver the insulin using a second device or an injection.

The input 110 is configured to receive sampled blood glucose data of the patient as part of the carbohydrate ratio test. The blood glucose data is obtained during a specified time duration. The specified time duration includes a time after delivery of the initial carbohydrate insulin bolus, but may include a time prior to the delivery of the initial carbohydrate insulin bolus as well. The configuration of the input 110 may depend on the type of BG management device 100. If the BG management device 100 is an insulin pump, the input 110 may be coupled to a GM included in the pump, or the input 110 may include a communication port to receive the blood glucose data from a second device. In some embodiments, the input 110 is coupled to the user interface 105, and the user may manually input the data into the pump through a keypad or keyboard included in the user interface. The blood glucose data may be received into a memory included with the controller 115 or separate from the controller 115.

If the BG management device 100 includes a GM, the input 110 may be coupled to blood glucose sensor circuit. The blood glucose sensor circuit includes a blood glucose sensor to produce a blood glucose signal representative of a blood glucose level of the patient. The blood glucose sensor circuit may include a sensor interface circuit to sample the blood glucose signal and may provide additional signal processing such as filtering for example. The blood glucose sensor circuit provides the sampled blood glucose data to the input 110. If the device includes neither a pump nor a GM, such as if the BG management device 100 is a computing device, the input 110 may include a communication port to receive the blood glucose data from a second device.

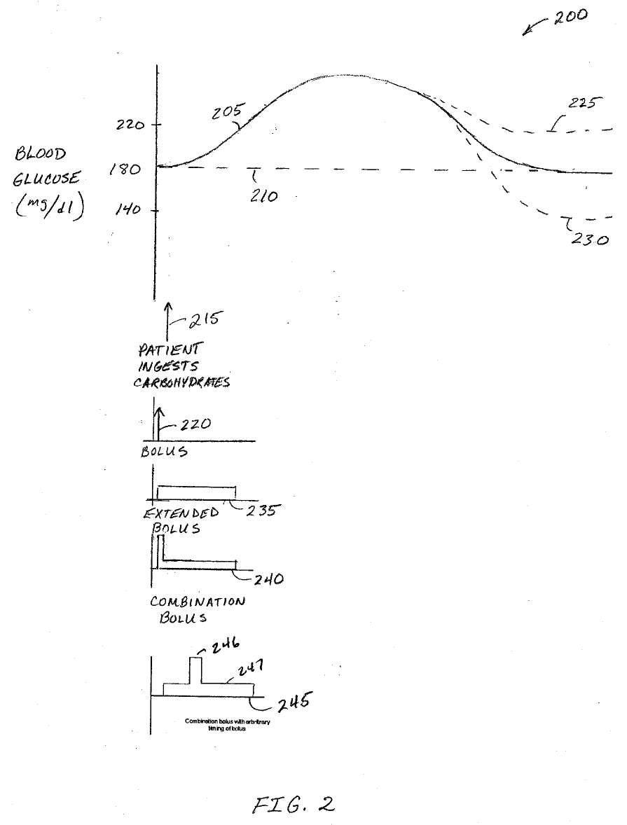

FIG. 2 is an example illustration of a graph 200 of blood glucose level during a carbohydrate ratio test. Assume, as shown in the waveform 205 of FIG. 2, that the blood glucose baseline level 210 is 180 mg/dl. The patient ingests a known amount of carbohydrates 215 as part of the test. An initial carbohydrate insulin bolus 220 is given in a time relationship to eating the carbohydrates. For example, if the BG management device 100 includes an insulin pump, the BG management device 100 may deliver the initial carbohydrate insulin bolus 220 a few minutes (e.g., 20 minutes) before instructing the patient to consume the carbohydrates. In other examples, the BG management device 100 may instruct the patient to initiate the delivery using a second device or to give themselves an injection. The waveform 205 shows that the blood glucose of the patient increases due to the ingested carbohydrates after a certain amount of time (e.g., one hour). The waveform 205 also shows that after a longer duration of time (e.g., three hours) the insulin reduces the blood glucose level of the patient back to the blood glucose baseline 210.

If the carbohydrate ratio is appropriate, the blood glucose level of the patient returns to the blood glucose baseline level 210 after the test, or within a specified range of the blood glucose baseline level 210 after the test. If the carbohydrate is not appropriate, the blood glucose level of the patient differs from the blood glucose baseline level 210 at the end of a specified duration as shown by the dashed-line waveforms 225, 230. The carbohydrate ratio module 120 determines a new carbohydrate ratio using the difference between the blood glucose level of the patient and the blood glucose baseline level 210 at the end of the specified time duration (e.g., three hours). In some embodiments, the difference from the blood glucose baseline level 210 may be required to exceed a threshold value before a new carbohydrate ratio is determined.

In some embodiments, if the blood glucose of the patient is above the blood glucose baseline level 210 at the end of the specified time duration (dashed-line waveform 225), the carbohydrate ratio module 120 determines an amount of correction insulin to reduce the blood glucose level of the patient to the blood glucose baseline. The carbohydrate ratio module 120 then determines the carbohydrate ratio by adding the correction insulin amount to the initial carbohydrate insulin amount, e.g.,

New Ratio=(Carbohydrates)/(initial insulin+correction insulin). (1)

For example, assume a current carbohydrate ratio for a patient is 10 grams per unit of insulin. As part of a carbohydrate ratio test, the patient is instructed to consume 50 grams of carbohydrates. An insulin calculation module 125 may calculate the initial carbohydrate insulin bolus to be 50/10 or 5 units of insulin. Also assume that after the specified time duration of the test, the blood glucose level of the patient stays 40 mg/dl above the established blood glucose baseline level 210 (e.g., 40 mg/dl higher than the baseline in FIG. 2, or 220 mg/dl). The current carbohydrate ratio appears to be too high and the carbohydrate insulin bolus delivered was too low.

Further assume that the correction factor for the patient is set to one unit per 80 mg/dl. A correction factor refers to the amount in drop in blood sugar, or blood glucose, for one unit of insulin. Using the correction factor, the carbohydrate ratio module 120 determines that 0.5 units of correction insulin [(40 mg/dl)/(80 mg/dl/unit)] are needed to reduce the blood glucose of the patient 40 mg/dl to the blood glucose baseline level 210. The carbohydrate ratio module 120 adds the correction amount to the initial carbohydrate ratio insulin bolus to determine that 5.5 units of insulin were needed to reduce the blood glucose level of the patient to the baseline level. The carbohydrate ratio module 120 calculates the new carbohydrate ratio to 50 grams/(5.0+0.5) units or 9.1 grams per unit of insulin.

In some embodiments, if the blood glucose of the patient is below the blood glucose baseline level 210 at the end of the specified time duration (dashed-line waveform 230), the carbohydrate ratio module 120 determines an amount of carbohydrate insulin that was over-delivered to the patient to reduce the blood glucose level of the patient to the blood glucose baseline. The carbohydrate ratio module 120 then determines the carbohydrate ratio by subtracting the over-delivered insulin amount from the initial carbohydrate insulin amount, e.g.,

New Ratio=(Carbohydrates)/(initial insulin−correction insulin). (2)

For example, again assume a current carbohydrate ratio for a patient is 10 grams per unit of insulin and the correction factor is 1 unit per 80 mg/dl. As part of a carbohydrate ratio test, the patient is instructed to consume 50 grams of carbohydrates. The initial carbohydrate insulin bolus is calculated to be (50 grams)/(10 grams/unit) or 5 units of insulin. This time however, the blood glucose level of the patient stays 40 mg/dl below the established blood glucose baseline level 210 after the specified time duration of the test (e.g., 40 mg/dl lower than the baseline in FIG. 2, or 140 mg/dl). The current carbohydrate ratio appears to be too low and the carbohydrate insulin bolus delivered was too high.

Using the correction factor of 1 unit per 80 mg/dl, the carbohydrate ratio module 120 determines that 0.5 units of carbohydrate insulin [(40 mg/dl)/(80 mg/dl/unit)] were over-delivered to the patient. The carbohydrate ratio module 120 subtracts the correction insulin amount from the initial carbohydrate ratio insulin bolus to determine that 4.5 units of insulin were needed to reduce the blood glucose level of the patient to the baseline level. The carbohydrate ratio module 120 calculates the new carbohydrate ratio to 50 grams/(5.0-0.5) units or 11.1 grams per unit of insulin.

The blood glucose level of the patient should be a reasonable amount above the target blood glucose level before a carbohydrate ratio test is executed in the device 100 to avoid a risk of going too low. The controller 115 may cancel the carbohydrate ratio test if a blood glucose level of the patient is outside of a specified range of blood glucose levels when the user wants to run the test. As another example, the user may elect to use a higher value for the current carbohydrate ratio to provide less risk of low blood glucose. In some embodiments, the insulin calculation module 125 is able to keep track of the amount of active insulin in the patient. This active insulin is sometimes referred to as insulin on board (IOB). To track the amount of active insulin, the insulin calculation module 125 uses the amount of insulin delivered, the time that elapsed since delivery of insulin and a duration of how long the insulin is active in the blood. The duration may be determined using kinetic action, which is the time it takes for insulin to disappear from the blood, or the duration of insulin action (DIA), which is how long the insulin lowers blood glucose. In some embodiments, the controller 115 may cancel the carbohydrate ratio test if active insulin is outside a specified range of active insulin.

The graphs 200 in FIG. 2 show a blood glucose level of a patient at a substantially constant level before the start of the carbohydrate ratio test. In some cases the blood glucose level of the patient may be changing before the start of the carbohydrate ratio test. In some embodiments, the controller 115 may cancel the carbohydrate ratio test if the rate of change of blood glucose of the patient is outside of a specified range of blood glucose level rates of change. If the blood glucose level is increasing or decreasing too fast prior to the test, the carbohydrate ratio test may not determine valid results or the test may result in an unsafe condition resulting from too high or too low a blood glucose level. In some examples, the controller 115 may cancel the carbohydrate ratio test if the blood glucose level of the patient is increasing (positive rate of change) at a rate faster than a specified rate, or if the blood glucose level of the patient is decreasing (negative rate of change) at a rate faster than a specified rate. In some embodiments, the controller 115 may cancel the carbohydrate ratio test according to a combination of blood glucose level and blood glucose rate of change. For example, the controller 115 may cancel the test if the blood glucose level of the patient is higher than a specified level and increasing at a faster rate than a specified rate, or if the blood glucose level of the patient is lower than a specified level and is decreasing at a rate faster than a specified rate.

In some embodiments, the carbohydrate ratio module 120 uses a rate of change of the blood glucose level in determining the carbohydrate ratio. In some embodiments, the carbohydrate ratio module 120 uses a table look-up method in determining the carbohydrate ratio. An example of a look-up table that includes rate of change of blood glucose is shown in Table 1 below. The left column of the table includes pre-defined ranges of rate of change of blood glucose measured in milligrams per deciliter per minute (mg/dl/min). The right column includes a multiplication factor used to adjust the insulin correction bolus based on the rate of change. The carbohydrate ratio module 120 calculates the amount of insulin over-delivered or under-delivered without the rate of change and then multiplies the calculated amount by the appropriate multiplication factor.

TABLE 1

| BG Rate of change | Correction bolus adjustment |
|---|---|
| >+3.0 mg/dl/min | +8% |
| Between +2.1 and +3.0 mg/dl/min | +6% |
| Between +1.1 and +2.0 mg/dl/min | +4% |
| Between +.1 and +1.0 mg/dl/min | +2% |
| <+/−.1 mg/dl/min | No adjustment |
| Between −0.1 and −1.0 mg/dl/min | −2% |
| Between −1.1 and −2.0 mg/dl/min | −4% |
| Between −2.1 and 3.0 mg/dl/min | −6% |
| >−3.0 mg/dl/min | −8% |

Recall the example above where after the specified time duration of the test, the blood glucose level of the patient stays 40 mg/dl above the established baseline level 210 (e.g., 40 mg/dl higher than the baseline in FIG. 2, or 220 mg/dl). In the example, it was determined that 0.5 units of insulin were under-delivered to the patient to cover the carbohydrates ingested and 0.5 units was the amount of the correction insulin used in equation (1). The carbohydrate ratio module 120 calculates the new carbohydrate ratio to 50 grams/5.5 units or 9.1 grams per unit of insulin. Now assume that the blood glucose level of the patient is increasing by 3.0 mg/dl/min. Using the Table, the carbohydrate ratio module 120 adjusts the correction insulin amount by +8% and calculates the new correction insulin amount to be (0.5)+(0.08)(0.5) or 0.54 units. The carbohydrate ratio module 120 calculates the new carbohydrate ratio to 50 grams/(5.0+0.54) units or 9.0 grams per unit of insulin.

In another example, assume that the blood glucose level of the patient is decreasing by 3.0 mg/dl/min. Using the Table, the carbohydrate ratio module 120 adjusts the correction insulin amount by −8% and calculates the new correction insulin amount to be (0.5)−(0.08)(0.5) or 0.46 units. The carbohydrate ratio module 120 calculates the new carbohydrate ratio to 50 grams/(5.0+0.46) units or about 9.2 grams per unit of insulin. Similar adjustments may be made using the look-up table for the case where an amount of insulin was over-delivered and the blood glucose level of the patient is increasing or decreasing after the specified time duration of the carbohydrate ratio test.

Taking into account the rate of change of blood glucose may allow shortening of the time duration carbohydrate ratio test because the BG management device may not have to wait as long to ensure that the patient's blood glucose level has settled. The blood glucose level rate of change can be used to end a carbohydrate ratio test early. In some embodiments, the carbohydrate ratio module 120 uses the rate of change of blood glucose to determine that the blood glucose level is stable, such as by when the rate of change is less than a specified threshold rate of change for example. The carbohydrate ratio module 120 may end a carbohydrate ratio test early and calculate a carbohydrate ratio when it determines that the blood glucose is stable. In some embodiments, the carbohydrate ratio module 120 may use the blood glucose rate of change to extrapolate to what the endpoint blood glucose level will be. For example, in FIG. 2, the measured blood glucose waveform 205 may be changing exponentially toward the final blood glucose level of 180 mg/dl. In some embodiments, the carbohydrate ratio module 120 may extrapolate that the final blood glucose level will be 180 mg/dl and end the carbohydrate ratio test early.

In some embodiments, the user interface 105 of FIG. 1 may include a display operatively coupled to the controller 115. The BG management device 100 may include a memory 116 communicatively coupled to the controller. The memory 116 may be the same memory that stores instructions executable by the controller 115, or may be a separate memory. The memory 116 stores a database of meal options in association with a known amount of carbohydrates. The controller 115 is configured to display a meal option from the database to the user. For example, if the patient is to consume 50 grams of carbohydrates as part of a carbohydrate ratio test, the BG management device 100 may suggest meal options that contain that amount of carbohydrates. In some embodiments, the meal option corresponds to an amount of carbohydrates that are packaged and prepared in such a way that the user can easily verify that they are eating the proper amount of carbohydrates. The user interface 105 receives the meal selection from the user.

In some embodiments, the memory 116 may store the database of meal options in association with a known amount of nutrient content. Nutrient content includes an amount of fat, protein, and carbohydrates in a meal option. The controller 115 may alter the carbohydrate insulin bolus profile based on the nutrient content of the meal option selection of the user.

FIG. 2 shows that the carbohydrate insulin bolus 220 may be delivered as a quick release of insulin. If the carbohydrate insulin bolus is to be delivered over an extended period of time, the insulin may be delivered as an extended bolus 235, or square-wave bolus. In some embodiments, insulin is delivered as a combination bolus 240 or dual wave bolus. A combination bolus 240 includes part of the insulin delivered quickly and part of the insulin delivered over an extended period of time as an extended bolus. Another combination bolus 245 shows that the quick delivery portion 246 can be delivered anywhere during the extended delivery portion 247. FIG. 2 shows that insulin can be delivered in a variety of patterns or profiles. One or more insulin delivery patterns may be configured into the controller 115 such as by software instructions. In some examples, the controller 115 uses the nutrient content of the meal option selection of the user when selecting a carbohydrate insulin bolus pattern. For example, fat slows down digestion and hence slows the digestion of carbohydrates. If the meal option selected by the user includes a high fat content, it may be preferable to deliver the carbohydrate insulin bolus over an extended period of time to match the slower digestion.

In some embodiments, the controller 115 executes multiple carbohydrate ratio tests according to a varying nutrient content of different meal options. The controller 115 displays a meal option with a known fat content. The user acknowledges the meal was consumed (e.g., through the user interface) and the controller executes a carbohydrate ratio test at least one time to determine a carbohydrate ratio that is appropriate for a meal of that fat content. The controller 115 may execute a number of carbohydrate ratio tests to determine carbohydrate ratios for meal options that have varying nutrient content. Once the carbohydrate ratio or ratios are determined, the controller 115 may change a carbohydrate bolus pattern or profile based on the nutrient content of a meal consumed.

For example, one carbohydrate ratio may work well for high carbohydrate, low fat or low protein meals but not for high fat or high protein meals. Using the blood glucose data, the controller 115 may determine that the blood glucose level of a patient goes low when the patient eats a type of meal with a certain nutrient content. The controller 115 may determine that the blood glucose is low when the blood glucose decreases below a specified blood glucose level (e.g., 70 mg/dl, or any other level specified by a clinician). The blood glucose may be going low because the patient is experiencing insulin resistance due to a higher amount of fat in the blood stream. The patient may need additional insulin when higher fat or higher protein meals are consumed. The controller 115 may use a lower carbohydrate ratio when the user indicates a higher fat or higher protein meal was eaten to provide the additional insulin.

In some embodiments, the controller 115 is configured to display user instructions for the carbohydrate ratio test. For example, the BG management device 100 may provide instructions, among other things, that the patient not eat for a period of time before the test begins, when to eat the carbohydrates, when to initiate delivery of the initial carbohydrate insulin bolus, not to initiate a correction bolus, and to maintain a normal activity level.

It may be desirable to use different carbohydrate ratios at different times during the day. For example, one carbohydrate ratio may be more appropriate during a time of day when the patient is less sensitive to insulin and another carbohydrate ratio may be more appropriate during a time of day when the patient is more sensitive to insulin. The BG management device 100 may include a timer circuit 117 operatively coupled to the controller 115. The controller 115 displays user instructions to determine a carbohydrate ratio at one or more specified times during a day. In some embodiments, controller 115 displays user instructions to run the carbohydrate ratio test on multiple days. The controller 115 may prompt the user to run the test during substantially the same time on the multiple days. This may result in more accurate carbohydrate ratios being used at different times during the day.

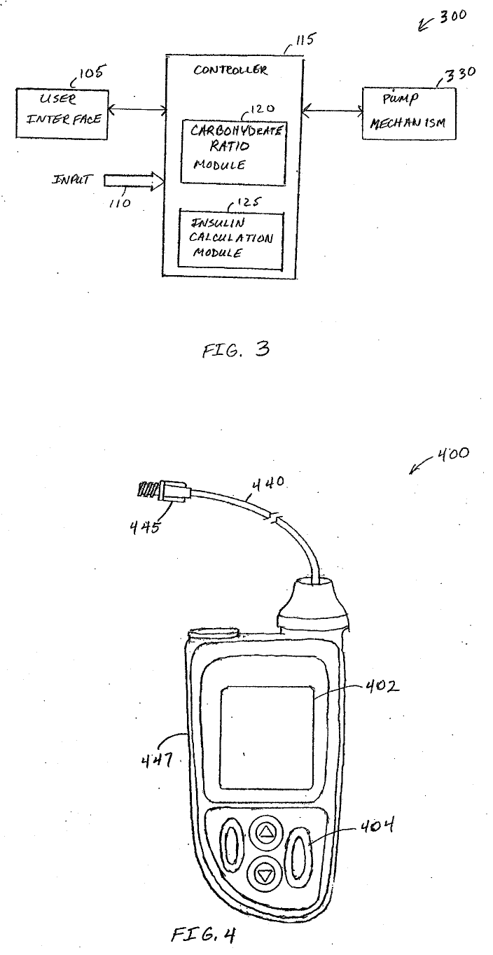

According to some embodiments, the BG management device 100 includes an insulin pump. FIG. 3 is a block diagram of portions of an example of a BG management device 300 that includes a pump mechanism 330 to deliver a carbohydrate bolus to the patient. The pump mechanism 330 is operatively coupled to the controller 115. The controller 115 may track the amount of insulin delivered via the pump mechanism 330. The insulin may be delivered through boluses such as a correction bolus or a carbohydrate bolus. The BG management device 300 may also deliver insulin according to a basal rate pattern or profile. In some examples, a basal rate pattern is stored in a memory included in the BG management device. If the initial carbohydrate insulin bolus is to be delivered according to bolus profile that includes an extended bolus, the carbohydrate ratio test may be executed longer by the controller 115 to account for the longer bolus delivery time.

In some embodiments, the insulin calculation module 125 is able to keep track of the amount of active insulin in the patient. This is sometimes referred to as insulin on board (IOB). To track the amount of active insulin, the controller 115 uses the amount of insulin delivered, the time that elapsed since delivery of insulin and a duration of how long the insulin is active in the blood. The duration may be determined using kinetic action, which is the time it takes for insulin to disappear from the blood, or the duration of insulin action (DIA), which is how long the insulin lowers blood glucose.

In some embodiments, the controller 115 cancels a carbohydrate ratio test if the insulin calculation module 125 determines that the active insulin amount is above a specified threshold amount. This is a conservative approach and minimizes the risk of IOB confounding the results of the carbohydrate ratio test.

As described above, the BG management device 300 may display instructions to not deliver a correction bolus during the carbohydrate ratio test. This is because the additional insulin will likely confound the test. In some embodiments, the controller 115 may suspend the start of the carbohydrate ratio test until the amount of active insulin becomes substantially zero. In some embodiments, the controller 115 may prevent delivery of a correction insulin bolus during the carbohydrate ratio test. For example, if the BG management device 300 includes a display, the controller 115 may not display an option of delivering a correction bolus. In another example, the controller 115 may cancel the carbohydrate ratio test if a delivery of a correction insulin bolus is detected during the carbohydrate ratio test. The controller 115 may display a recommendation that the correction bolus not be delivered. If the user elects to deliver the correction bolus despite the recommendation, the controller 115 may cancel the carbohydrate ratio test.

FIG. 4 is an illustration of a BG management device 400 that includes an insulin pump mechanism. The BG management device 400 includes a cassette or cartridge of insulin and tubing 440 connectable to a patient such as by a Luer lock 445. The BG management device 400 includes a user interface that may include a display 402 in electrical communication with a controller 115. The user interface may also include one or more keys 404.

Returning to FIG. 3, the blood glucose data may be produced by a second device separate from the BG management device 300. The controller 115 displays user instructions for the determination of the carbohydrate ratio. The user interface 105 and the input 110 are configured to receive the sampled blood glucose data entered manually by the user through the user interface 105. The controller 115 may periodically prompt the user to enter a blood glucose value at different times during the test, or to enter the blood glucose data all at once after the test. The prompt to enter a blood glucose value may be included with the displayed instructions discussed previously.

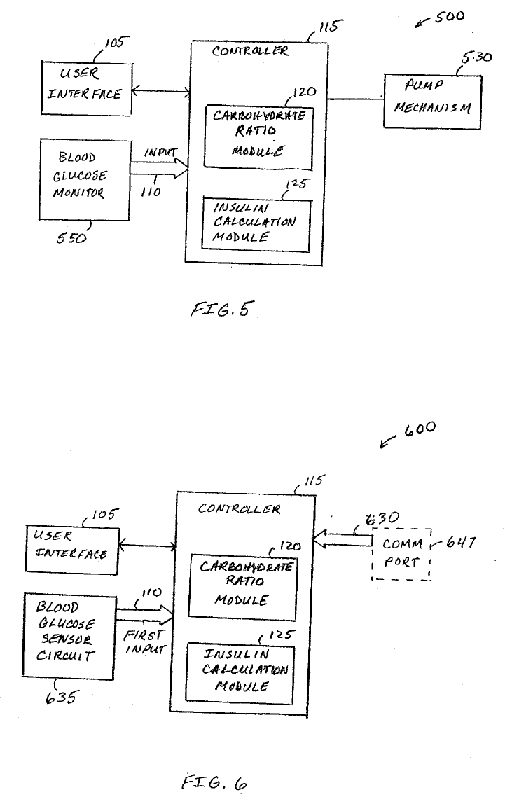

FIG. 5 is another block diagram of portions of a BG management device 500 that includes a pump mechanism 530. A blood glucose monitor, or GM 550, is communicatively coupled to the input 110. The input 110 is configured to receive the sampled blood glucose data from the GM 550. In some examples, the GM 550 is included in the BG management device 500 and is coupled to the input. In some examples, the GM 550 is included in a second device. The input 110 may include a communication port, such as communication port 447 located on the rear face of the device in FIG. 4, and the GM 550 is communicatively coupled to the input 110 by the communication port 447. In some embodiments, the communication port 447 includes a wired port such as a serial interface or bus interface for communicating with the second device. In some embodiments, the communication port 447 includes a wireless port such as an infrared (IR) communication port or a radio frequency (RF) communication port. The input wirelessly receives the sampled blood glucose data from the second device.

Returning to FIG. 5, in some embodiments, the included GM 550 is a continuous GM and automatically collects the sampled blood glucose data. For example, the GM 550 may include a blood glucose sensor. The blood glucose sensor produces a blood glucose signal representative of a blood glucose level of the patient. The GM 550 samples the blood glucose signal to obtain the sampled blood glucose data. With a continuous GM, the carbohydrate ratio test runs automatically after a user prompt is received that begins the test. The blood glucose measurements are automatically made by the continuous GM.

According to some embodiments, the user may need to prompt the GM 550 to begin a blood glucose measurement. For example, the GM 550 may require diabetes test strips to take a blood glucose measurement. The controller 115 prompts the user, via a display, to begin a blood glucose measurement using the GM 550. The user then provides a new test strip to the GM 550 when prompted during the carbohydrate ratio test. In another example, the GM 550 may include a drum of diabetes test strips and the user advances the drum to a fresh or unused test strip when prompted by the controller 115. The controller 115 may display the determined carbohydrate ratio after the carbohydrate ratio test. The controller 115 may also communicate the carbohydrate ratio to the second device via the communication port.

According to some embodiments, the carbohydrate ratio module 120 may determine that a blood glucose level of the patient decreased below the blood glucose baseline substantially near the beginning of the specified time duration of the carbohydrate ratio test. For example, the blood glucose level may decrease below the blood glucose baseline within the first hour after delivery of the initial carbohydrate insulin bolus. The controller 115 produces an indication to the user recommending a second carbohydrate ratio test, such as via a display for example. During the second carbohydrate ratio test, the controller 115 delivers a carbohydrate insulin bolus that includes an extended insulin bolus. In some embodiments, the extended insulin bolus is included in a combination bolus. The carbohydrate ratio module 120 determines the carbohydrate ratio according to a difference between the blood glucose baseline and a blood glucose level of the patient at the end of a specified time duration of the second carbohydrate ratio test.

As described above, the BG management device 500 may include a memory to store a database of meal options in association with a known amount of nutrient content. The user then selects a meal option before beginning a carbohydrate ratio test. If the blood glucose level of the patient goes low, the controller 115 may alter a carbohydrate insulin bolus profile based on the nutrient content of the meal option selection of the user and on the received blood glucose data.

For example, if the patient selects and eats a high carbohydrate, low fat meal and the blood glucose data indicates that the patient's blood glucose level goes low soon after the carbohydrate bolus (e.g., within 1 hour), the patient may have delayed gastric-emptying also known as gastroparesis. Gastroparesis often slows the absorption of food after meals. Low blood sugar may occur soon after the meal because the insulin in the carbohydrate bolus is acting before the food is absorbed. Based on the meal option selection and the obtained blood glucose data, the controller 115 may alter a carbohydrate insulin bolus profile to reduce the risk of a low blood glucose level. For example, the controller 115 may alter a carbohydrate bolus given in the course of a carbohydrate test, such as by using a bolus profile or pattern that includes an extended bolus during the test for example. In FIG. 2, if a combination bolus 245 is used, the timing of the quick delivery portion 246 of the combination bolus can be timed to the patient's gastric emptying. Beyond the time of the test, the controller 115 may only enable carbohydrate insulin bolus profiles that include an extended bolus alone or in a combination bolus. In some embodiments, the controller 15 may recommend (e.g., using a display) at least one bolus profile that includes an extended bolus to the user, and the user is given the option of accepting or rejecting the recommended profile.

In another example, if the patient selects and eats a high fat meal and the blood glucose data indicates that the patient's blood glucose level goes low soon after the carbohydrate bolus, the patient may need a carbohydrate bolus that includes an extended bolus after eating those types of meals. In some embodiments, the controller 15 may automatically select a carbohydrate insulin bolus profile that includes an extended bolus when the user selects a high fat meal option. In some embodiments, the controller 15 may recommend at least one bolus profile that includes an extended bolus to the user when the user indicates a meal option, and the user is given the option of accepting or rejecting the recommended profile.

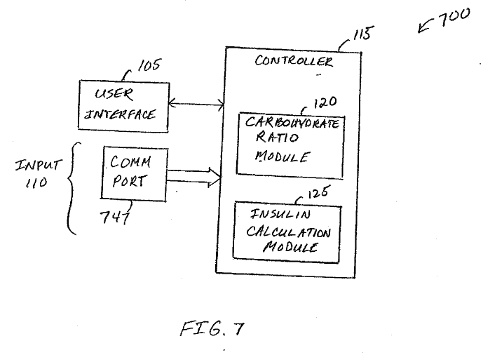

According to some embodiments, the BG management device is a GM. FIG. 6 is a block diagram of a BG management device 600 that includes a blood glucose sensor circuit 635 operatively coupled to the input 110. The blood glucose sensor circuit 635 produces a blood glucose signal representative of a blood glucose level of the patient and provides the sampled blood glucose data to input 110. In some embodiments, the blood glucose sensor circuit 635 includes an implantable blood glucose sensor. In some embodiments, the blood glucose sensor includes a percutaneous blood glucose sensor. The blood glucose sensor circuit 635 may include signal conditioning circuits, such as for signal filtering and signal amplification for example. If an implantable blood glucose sensor is used, the blood glucose sensor circuit 635 may include a communication circuit configured to receive blood glucose data wirelessly, such as by RF communication.

The BG management device 600 includes a second input 630 in electrical communication with the controller 115. The second input 630 receives information related to insulin delivery. The information may include one or more of an amount of insulin in the initial carbohydrate insulin bolus, a carbohydrate ratio, and an amount of active insulin, if any, in the patient. The information related to insulin delivery may be received into a memory. The carbohydrate ratio module 120 determines the carbohydrate ratio using the insulin delivery information and the sampled blood glucose data.

The BG management device 600 may include a communication port 647 coupled to the second input 630. The communication port 647 receives the information related to insulin delivery from a second device. In some embodiments, the communication port 647 includes a wired port such as a serial interface or bus interface. In some embodiments, the communication port 647 includes a wireless port such as an infrared (IR) communication port or a radio frequency (RF) communication port. The second input 630 wirelessly receives the insulin delivery data from the second device. As an example, the second device may be an insulin pump. The controller 115 is configured for communicating the carbohydrate ratio through the communication port 647 or may display the carbohydrate ratio on a display. In some embodiments, the BG management device may calculate the amount of insulin in the initial carbohydrate insulin bolus using the information related to insulin delivery and communicate the initial bolus amount, such as by a display or through the communication port for example.

In some embodiments, the user interface 105 and the second input 630 are configured to receive the information related to insulin delivery by a user manually entering the information through the user interface 105. The insulin delivery information may be obtained from a pump or may be information associated with insulin delivered by injection, such as from MDI therapy for example. The controller 115 may display the carbohydrate ratio.

FIG. 7 is a block diagram of portions of another example of a BG management device 700. BG management device 700 includes neither a GM nor an insulin pump. For example, the BG management device 700 may be a computing device such as a personal computer or personal data assistant (PDA) to assist the patient in managing insulin therapy. The BG management device 700 includes a user interface 105, an input 110, and a controller 115 in electrical communication with the input 110 and the user interface 105. The input 110 includes at least one communication port 747 configured for receiving sampled blood glucose information. The communication port 747 may provide a wired connection to a second device, or the communication port 747 may provide a wireless connection to a second device. The sampled blood glucose information may include at least one time-stamp in order to align the sampled blood glucose information to information related to insulin delivery.

The insulin delivery information may be received through the same communication port 747 or a second communication port. The sampled blood glucose information and the insulin delivery information may be received into a memory. The communication ports may be any combination of wired or wireless communication ports. The information may include one or more of an amount of insulin in the initial carbohydrate insulin bolus, a time the carbohydrate insulin bolus was delivered, a carbohydrate ratio, and an amount of active insulin, if any, in the patient. The insulin delivery information may include at least one time-stamp to align the insulin delivery information with the blood glucose information. The time stamp may correspond to the time the carbohydrate insulin bolus was delivered or indicate a different time. The controller 115 may communicate the carbohydrate ratio through the communication port and/or the controller 115 may display the carbohydrate ratio. In some embodiments, the BG management device 700 may calculate the amount of insulin in the initial carbohydrate insulin bolus using a current carbohydrate ratio and communicate the insulin amount via a communication port to another device or via a display.

### Method Embodiments

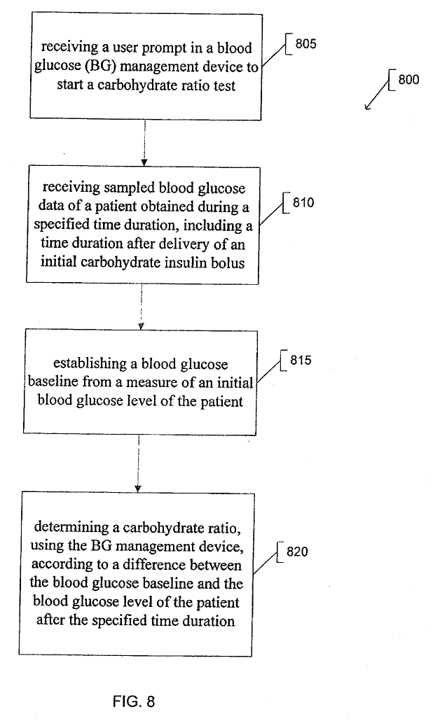

FIG. 8 is a flow diagram of a method 800 of automatically determining a carbohydrate ratio using blood glucose data. At block 805, a user prompt is received into a BG management device to start a carbohydrate ratio test. The user interface may include a push-button, keypad, or mouse. The user interface may also include a display to display one or more instructions for the user to execute the test, and to display a carbohydrate ratio.

At block 810, sampled blood glucose data is received in the BG management device. The blood glucose data is obtained from a patient during a specified time duration, including a time after delivery of an initial carbohydrate insulin bolus. At block 815, a blood glucose baseline is established from one or measures of a blood glucose level of a patient. In some embodiments, a baseline is established using an average of multiple blood glucose measurements. At block 820, the carbohydrate ratio is determined using the BG management device according to a difference between the blood glucose baseline and the blood glucose level of the patient after the specified time duration.

In some embodiments, if the blood glucose of the patient is above the blood glucose baseline at the end of the specified time duration (i.e. there was an under-delivery of an amount of insulin to cover the carbohydrates), the method 800 includes determining an amount of correction insulin to reduce the blood glucose of the patient to the blood glucose baseline. The carbohydrate ratio is determined by adding the correction insulin amount and the initial carbohydrate insulin bolus amount.

In some embodiments, if the blood glucose of the patient is below the blood glucose baseline at the end of the specified time duration (i.e. there was an over-delivery of an amount of insulin to cover the carbohydrates), the method 800 includes determining an amount of insulin over-delivered to the patient. The carbohydrate ratio is determined by subtracting the over-delivered insulin amount from the initial carbohydrate insulin bolus amount.

In some embodiments, the method 800 includes determining the carbohydrate ratio using a rate of change of the blood glucose level of the patient as indicated by the blood glucose data.

According to some embodiments, receiving the user prompt includes receiving an electrical signal via a user interface of the BG management device to start the determination of the carbohydrate ratio and providing instructions to a user for the determination using the BG management device. The instructions may be provided using a BG management device display.

In some embodiments, the method 800 includes presenting a user with at least one meal option from a database in a BG management device before delivery of the carbohydrate insulin bolus. At least one meal option is displayed by the BG management device. The meal option or options are associated in the database with a known amount of carbohydrates. This assists the user in easily identifying food or meals that have the necessary amount of carbohydrates to be ingested before the test. An amount of insulin in the initial carbohydrate insulin bolus is calculated based on an initial carbohydrate ratio value and an amount of carbohydrates ingested by the patient. In some embodiments, a meal option is associated in the database with a known amount of nutrient content. The method 800 may include altering a pattern or profile of delivery of the initial carbohydrate insulin bolus based on a meal option selected. For example, the nutrient content of a meal option may indicate that the carbohydrate insulin bolus is better delivered using an extended bolus or a combination bolus, such as if a meal option selected by a user includes a high amount of fat.

In some embodiments, the method 800 includes executing multiple carbohydrate ratio tests according to a varying nutrient content of different meal options. In this way, a patient's reaction to the amount of fat, protein, or carbohydrates can be determined. A meal option having a known nutrient content is displayed. If the user acknowledges the meal was consumed, a carbohydrate ratio test is run to determine a carbohydrate ratio that is appropriate for a meal of that nutrient content. A number of carbohydrate ratio tests may be run to determine carbohydrate ratios for varying nutrient content, such as a varying amount of fat for example. Once the carbohydrate ratios are determined, a carbohydrate bolus pattern or profile based on the nutrient content of a meal consumed is determined. For example, one carbohydrate ratio may work well for high carbohydrate, low fat or low protein meals but not for high fat or high protein meals. For example, the patient may be experiencing insulin resistance due to a higher amount of fat in the blood stream. The patient may need additional insulin when higher fat or higher protein meals are consumed. The controller 115 may use a lower carbohydrate ratio when the user indicates a higher fat or higher protein meal was eaten to provide the additional insulin.

According to some embodiments, the method 800 includes delivering the initial insulin carbohydrate bolus using the BG management device, i.e. the BG management device includes a pump mechanism to deliver insulin. In some embodiments, method 800 includes determining an amount of active insulin (IOB) in the patient prior to delivering the initial insulin carbohydrate bolus. If an amount of active insulin is above a specified threshold active insulin amount, the BG management device may cancel the carbohydrate ratio test. In some examples, the method 800 includes preventing the BG management device from delivering a correction bolus of insulin during the carbohydrate ratio test. In some examples, the method 800 includes canceling the carbohydrate ratio test if a correction bolus of insulin is delivered during the carbohydrate ratio test.

According to some embodiments, the method 800 includes determining that a blood glucose level of the patient decreases below the blood glucose baseline substantially near the beginning of the specified time duration using the BG management device, indicating to a user that a second determination of the carbohydrate ratio is recommended, and delivering a carbohydrate insulin bolus that includes an extended insulin bolus during the second determination of the carbohydrate ratio. The extended bolus may be included in a combination bolus.

In some embodiments, the method 800 includes displaying one or more meal options for the patient to consume before beginning a carbohydrate ratio test. A carbohydrate insulin bolus profile may be altered based on the nutrient content of the meal selection of the user and on the received blood glucose data if the blood glucose level of the patient goes low during the test. The altered carbohydrate insulin bolus profile may include an extended bolus. In some embodiments, the controller 115 may recommend (e.g., using a display) at least one bolus profile that includes an extended bolus to the user, and the user is given the option of accepting or rejecting the recommended profile.

According to some embodiments, the BG management device includes an insulin pump and a GM. The method 800 includes automatically receiving the sampled blood glucose data from the blood glucose monitor. In some embodiments, the BG management device includes the insulin pump and the blood glucose data is obtained using a separate device. The method 800 includes receiving the sampled blood glucose data into the BG management device from the separate device through a communication port. The communication port may be a wireless communication port and the data is received wirelessly, or the communication port can be a wired port. The separate device may be a continuous GM. With a continuous GM, the carbohydrate ratio test runs automatically after a user prompt is received that begins the test. The blood glucose measurements are automatically made by the continuous GM.

In some embodiments, the separate device may be a GM that requires some action by the user to obtain a blood glucose reading. For example, the GM may require the user to place a test strip into the GM in order to obtain a glucose reading. In some embodiments, the method 800 may include prompting the user through a user interface to obtain blood glucose data using the separate device. The prompting may be periodic during the carbohydrate ratio test.

In some embodiments, the blood glucose data obtained from the separate device is entered manually into the BG management device. The method 800 includes the BG management device receiving the blood glucose data through the user interface. The user interface is configured for manual entry of blood glucose data, such as by including a keypad and a display. The user reads the blood glucose data from the separate GM and manually enters the blood glucose data into the BG management device. In some embodiments, the method 800 includes the BG management device periodically prompting the user to manually enter a blood glucose value during the carbohydrate ratio test.

According to some embodiments, the BG management device includes a GM and does not include an insulin pump. A carbohydrate insulin bolus is delivered using a second separate device. The sampled blood glucose data is received automatically using the included GM. The method 800 further includes receiving information related to insulin delivery into the BG management device from the separate device into the BG management device. In some embodiments, the information related to insulin delivery includes the amount of insulin in the carbohydrate insulin bolus.

According to some embodiments, the method 800 includes receiving the insulin delivery information into the BG management device through a communication port. In some embodiments, the BG management device may calculate the initial carbohydrate insulin bolus amount using a current carbohydrate ratio.

After a carbohydrate ratio test, the BG management device may communicate the carbohydrate ratio to the separate device using the communication port. This is useful if the separate device is an insulin pump. In some embodiments, the method 800 includes receiving the insulin delivery information into the BG management device by manually entering the insulin delivery information. The information is manually entered via a user interface on the BG management device. The carbohydrate ratio may be displayed on the BG management device after the carbohydrate ratio test.

According to some embodiments, the BG management device does not include a BG monitor or an insulin pump. The initial carbohydrate insulin bolus is delivered using a second separate device, such as an insulin pump for example. The method 800 includes providing insulin delivery information, such as the amount insulin in the carbohydrate insulin bolus to the BG management device using the second device. The insulin delivery information may also include a correction factor, carbohydrate ratio, and an amount of active insulin in the patient.

In some embodiments, the BG management device may calculate the initial carbohydrate insulin bolus amount using a current carbohydrate ratio and display an initial carbohydrate insulin bolus amount or communicate the amount to the second device. The BG management device receives sampled blood glucose data from the second separate device or a third device. At least one of the insulin delivery information and the sampled blood glucose data includes a time-stamp to allow for alignment of the insulin delivery information and the blood glucose data. For example, the time-stamp for the insulin delivery may be the carbohydrate insulin bolus delivery time. The carbohydrate ratio is determined using the sampled blood glucose data and the insulin delivery information. The updated carbohydrate ratio may be displayed or communicated to the second device.

The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations, or variations, or combinations of various embodiments. Combinations of the above embodiments, and other embodiments not specifically described herein, will be apparent to those of skill in the art upon reviewing the above description.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own.

What is claimed is:

1. An apparatus comprising:
a user interface configured to generate an electrical signal to begin a carbohydrate ratio test when prompted by a user;
an input configured to receive sampled blood glucose data of a patient that is obtained during a specified time duration, including a time duration after delivery of an initial carbohydrate insulin bolus; and
a controller in electrical communication with the input and the user interface, the controller including a carbohydrate ratio module configured to:
establish a blood glucose baseline from a measure of an initial blood glucose level of the patient; and
determine a carbohydrate ratio according to a difference between a blood glucose level of the patient at the end of the specified time duration and the blood glucose baseline.

2. The apparatus of claim 1, wherein the carbohydrate ratio module is further configured to:
determine an amount of correction insulin to reduce the blood glucose of the patient to the blood glucose baseline if the blood glucose of the patient is above the blood glucose baseline at the end of the specified time duration; and
determine the carbohydrate ratio by adding the correction insulin amount to the initial carbohydrate insulin amount.

3. The apparatus of claim 1, wherein the carbohydrate ratio module is further configured to:
determine an amount of insulin that was over-delivered to the patient if the blood glucose of the patient is below the blood glucose baseline at the end of the specified time duration; and
determine the carbohydrate ratio by subtracting the over-delivered insulin amount from the initial carbohydrate insulin bolus amount.

4. The apparatus of claim 1, wherein the carbohydrate ratio module is configured to determine a carbohydrate ratio according to a difference between a blood glucose level of the patient at the end of the specified time duration and the blood glucose baseline and a rate of change of the blood glucose level of the patient.

5. The apparatus of claim 1, further including an insulin calculation module configured to calculate the initial carbohydrate insulin bolus based on an initial carbohydrate ratio and an amount of carbohydrates ingested by the patient.

6. The apparatus of claim 5, further including:
a display in electrical communication with the controller; and
a memory configured to store a database of meal options in association with a known amount of carbohydrates,
wherein the controller is configured to display a meal option from the database to the user, and
wherein the user interface is further configured to receive a meal selection from the user to indicate the amount of carbohydrates.

7. The apparatus of claim 6, wherein the memory is further configured to store the database of meal options in association with a known amount of nutrient content.

8. The apparatus of claim 7, further including:
a pump mechanism, operatively coupled to the controller, configured to deliver the initial carbohydrate insulin bolus to the patient according to a specified bolus delivery profile;
wherein the carbohydrate ratio module is configured to determine that a blood glucose level of the patient decreased below a specified blood glucose threshold substantially near the beginning of the specified time duration; and
wherein the controller is configured to alter the bolus delivery profile according to the blood glucose data and the nutrient content of meal option selection received from the user.

9. The apparatus of claim 6, wherein the controller is configured to display at least one user instruction for the carbohydrate ratio test.

10. The apparatus of claim 5, further including:
a pump mechanism configured to deliver the initial carbohydrate insulin bolus to the patient, wherein the pump mechanism is operatively coupled to the controller; and
a blood glucose monitor communicatively coupled to the input.

11. The apparatus of claim 10, wherein the blood glucose monitor is a continuous blood glucose monitor configured to automatically collect the sampled blood glucose data.

12. The apparatus of claim 10, further including:
a display in electrical communication with the controller, and
wherein the controller is configured to prompt the user, via the display, to begin a blood glucose measurement using the blood glucose monitor.

13. The apparatus of claim 5, further including:
a pump mechanism configured to deliver the initial carbohydrate insulin bolus to the patient, wherein the pump mechanism is operatively coupled to the controller; and
wherein the user interface and the input are configured to receive the sampled blood glucose data entered manually by the user.

14. The apparatus of claim 13, further including:
a display in electrical communication with the controller, wherein the controller is configured to display at least one user instruction for the carbohydrate ratio test, including periodically prompting the user to enter a blood glucose value.

15. The apparatus of claim 5, further including:
a pump mechanism configured to deliver the initial carbohydrate insulin bolus to the patient, wherein the pump mechanism is operatively coupled to the controller;
wherein the insulin calculation module is further configured to determine an amount of active insulin in the patient, and
wherein the controller is configured to cancel the carbohydrate ratio test if the active insulin amount is above a specified threshold amount.

16. The apparatus of claim 5, further including:
a pump mechanism configured to deliver the initial carbohydrate insulin bolus to the patient, wherein the pump mechanism is operatively coupled to the controller; and
wherein the controller is configured to prevent delivery of a correction insulin bolus during the carbohydrate ratio test.

17. The apparatus of claim 5, further including:
a pump mechanism configured to deliver the initial carbohydrate insulin bolus to the patient, wherein the pump mechanism is operatively coupled to the controller; and
wherein the controller is configured to cancel the carbohydrate ratio test if a delivery of a correction insulin bolus is detected during the carbohydrate ratio test.

18. The apparatus of claim 5, further including:
a pump mechanism configured to deliver the initial carbohydrate insulin bolus to the patient, wherein the pump mechanism is operatively coupled to the controller;
wherein the carbohydrate ratio module is configured to determine that a blood glucose level of the patient decreased below the blood glucose baseline substantially near the beginning of the specified time duration, and
wherein the controller is configured to:
produce an indication recommending a second carbohydrate ratio test; and
deliver a carbohydrate insulin bolus, including an extended insulin bolus, during the second carbohydrate ratio test; and
wherein the carbohydrate ratio module is further configured to determine the carbohydrate ratio according to a difference between the blood glucose baseline and a blood glucose level of the patient at the end of a specified time duration of the second carbohydrate ratio test.

19. The apparatus of claim 1, wherein the controller is configured to cancel the carbohydrate ratio test if a blood glucose level of the patient is outside of a specified range of blood glucose levels.

20. The apparatus of claim 1, wherein the controller is configured to cancel the carbohydrate ratio test if the rate of change of blood glucose of the patient is outside of a specified range of blood glucose level rates of change.

21. The apparatus of claim 1, further including:
a timer circuit; and
a display, wherein the timer circuit and the display are operatively coupled to the controller, and wherein the controller is configured to display at least one user instruction for executing the carbohydrate ratio test at one or more specified times during a day.

22. The apparatus of claim 21, wherein the controller is configured to display the user instruction during a substantially same time on multiple days.

23. The apparatus of claim 1, further including a display in electrical communication with the controller, and wherein the controller is configured to display the carbohydrate ratio.

24. The apparatus of claim 1, wherein the input is a first input and the apparatus further includes:
a blood glucose sensor circuit operatively coupled to the first input, the blood glucose sensor circuit configured to produce a blood glucose signal representative of a blood glucose level of the patient and provide the sampled blood glucose data to the first input;
a second input in electrical communication with the controller, wherein the controller is configured to receive information related to insulin delivery via the second input; and
wherein the carbohydrate ratio module is configured to determine the carbohydrate ratio using the insulin delivery information and the sampled blood glucose data.

25. The apparatus of claim 24, wherein the information related to insulin delivery includes:
an amount of insulin in a carbohydrate insulin bolus;
a carbohydrate ratio; and
an amount of active insulin, if any, in the patient.

26. The apparatus of claim 24, further including a communication port coupled to the second input, the communication port to receive the information related to insulin delivery.

27. The apparatus of claim 26, wherein the controller is configured to communicate the carbohydrate ratio through the communication port.

28. The apparatus of claim 24, wherein the user interface and the second input are configured to receive the information related to insulin delivery entered manually by the user.

29. The apparatus of claim 1, wherein the input includes a communication port configured to receive the sampled blood glucose information together with at least one time-stamp and to receive information related to insulin delivery, including an amount of insulin in the carbohydrate insulin bolus and a time the carbohydrate insulin bolus was delivered, and
wherein the carbohydrate ratio module is configured to determine the carbohydrate ratio using the information related to insulin delivery and the time-stamped sampled blood glucose data.

30. The apparatus of claim 29, wherein the information related to insulin delivery includes:
an amount of insulin in the carbohydrate insulin bolus;
a time the carbohydrate insulin bolus was delivered;
a carbohydrate ratio; and
an amount of active insulin, if any, in the patient.

31. The apparatus of claim 29, wherein the controller is configured to communicate the carbohydrate ratio through the communication port.

32. A method comprising:
receiving a user prompt in a blood glucose (BG) management device to start a carbohydrate ratio test;
receiving sampled blood glucose data of a patient obtained during a specified time duration, including a time duration after delivery of an initial carbohydrate insulin bolus;
establishing a blood glucose baseline from at least one measure of a blood glucose level of the patient; and
determining a carbohydrate ratio, using the BG management device, according to a difference between the blood glucose baseline and the blood glucose level of the patient after the specified time duration.

33. The method of claim 32, further including:
determining an amount of correction insulin to reduce the blood glucose of the patient to the blood glucose baseline if the blood glucose of the patient is above the blood glucose baseline at the end of the specified time duration; and
determining the carbohydrate ratio by adding the correction insulin amount and the initial carbohydrate insulin bolus amount.

34. The method of claim 32, further including:
determining an amount of insulin that was over-delivered to the patient if the blood glucose of the patient is below the blood glucose baseline at the end of the specified time duration; and
determining the carbohydrate ratio by subtracting the over-delivered insulin amount from the initial carbohydrate insulin bolus amount.

35. The method of claim 32, wherein determining the carbohydrate ratio includes determining the carbohydrate ratio using a rate of change of the blood glucose level of the patient.

36. The method of claim 32, further including:
displaying at least one meal option from a database in the BG management device before delivery of the carbohydrate insulin bolus, the meal option associated in the database with a known amount of carbohydrates;
receiving a meal option selection in the BG management device; and
calculating an amount of the initial carbohydrate insulin bolus based on an initial carbohydrate ratio value and an amount of carbohydrates in the meal option selection.

37. The method of claim 36, wherein displaying at least one meal option includes presenting a user with at least one meal option associated in the database with a known amount of nutrient content.

38. The method of claim 37, including:
determining, using the blood glucose data, that a blood glucose level of the patient decreased below a specified blood glucose level after delivery of the initial carbohydrate insulin bolus, the carbohydrate insulin bolus delivered according to a bolus delivery profile; and
altering the bolus delivery profile using the blood glucose data and a meal selection received from a user.

39. The method of claim 32, further including:
receiving an electrical signal via a user interface of the BG management device to start a determination of the carbohydrate ratio; and
displaying at least one instruction to a user for the determination using the BG management device.

40. The method of claim 32, including delivering the initial insulin correction bolus using the BG management device.

41. The method of claim 40, further including:
determining an amount of active insulin in the patient prior to delivering the initial carbohydrate insulin bolus; and
canceling the carbohydrate ratio test if an amount of active insulin is above a specified threshold active insulin amount.

42. The method of claim 40, further including preventing the BG management device from delivering a correction bolus of insulin during the carbohydrate ratio test.

43. The method of claim 40, further including canceling the carbohydrate ratio test if a correction bolus of insulin is delivered during the carbohydrate ratio test.

44. The method of claim 40, further including:
determining that a blood glucose level of the patient decreases below the blood glucose baseline substantially near the beginning of the specified time duration using the BG management device;
indicating that a second determination of the carbohydrate ratio is recommended; and
delivering a carbohydrate insulin bolus that includes an extended insulin bolus during the second determination of the carbohydrate ratio.

45. The method of claim 40, wherein receiving sampled blood glucose data includes automatically receiving the sampled blood glucose data from a blood glucose monitor included in the BG management device, and wherein the method includes automatically running the carbohydrate ratio test using the BG management device after the user prompt is received.

46. The method of claim 40, wherein receiving sampled blood glucose data includes:
obtaining the sampled blood glucose data using a device separate from the BG management device; and
receiving the sampled blood glucose data into the BG management device from the separate device through a communication port.

47. The method of claim 46, wherein receiving sampled blood glucose data includes wirelessly receiving the sampled blood glucose data into the BG management device from the separate device through a wireless communication port.

48. The method of claim 47, wherein receiving sampled blood glucose data includes periodically prompting a user through a user interface of the BG management device to obtain blood glucose data using the separate device.

49. The method of claim 40, wherein receiving sampled blood glucose data includes receiving the sampled blood glucose data through a user interface of the BG management device configured for manual entry of blood glucose data.

50. The method of claim 49, wherein receiving sampled blood glucose data includes prompting a user to manually enter a blood glucose value during the carbohydrate ratio test.

51. The method of claim 32, including delivering the initial carbohydrate insulin bolus using a second device;
wherein receiving sampled blood glucose data includes automatically receiving the sampled blood glucose data from a blood glucose monitor included in the BG management device; and
wherein the method further includes receiving information related to insulin delivery into the BG management device.

52. The method of claim 51, wherein the information related to insulin delivery includes:
an amount of insulin in the initial carbohydrate insulin bolus;
a carbohydrate ratio; and
an amount of active insulin, if any, in the patient.

53. The method of claim 51, wherein receiving the information related to insulin delivery includes receiving the information related to insulin delivery from the second device through a communication port.

54. The method of claim 53, further including communicating the carbohydrate ratio to the second device using the communication port.

55. The method of claim 51, wherein receiving the information related to insulin delivery includes receiving the information related to insulin delivery manually through a user interface on the BG management device.

56. The method of claim 51, further including displaying the carbohydrate ratio using the BG management device.

57. The method of claim 32, further including:
delivering the initial carbohydrate insulin bolus using a second device;
providing information related to the initial carbohydrate insulin bolus to the BG management device using the second device,
wherein receiving sampled blood glucose data includes receiving time-stamped sampled blood glucose data into the BG management device, and
wherein determining the carbohydrate ratio includes determining the carbohydrate ratio using the time-stamped sampled blood glucose data and the information related to the initial carbohydrate insulin bolus.

58. The method of claim 57, wherein the information related to insulin delivery includes:
an amount of insulin in the initial carbohydrate insulin bolus;
a bolus delivery time;
a carbohydrate ratio; and
an amount of active insulin, if any, in the patient.

59. An apparatus comprising:
means for receiving a user prompt in a blood glucose (BG) management device to start a carbohydrate ratio test;
means for receiving sampled blood glucose data of a patient obtained during a specified time duration, including a time duration after delivery of an initial carbohydrate insulin bolus;
means for establishing a blood glucose baseline from a measure of an initial blood glucose level of the patient; and
means for determining a carbohydrate ratio, using the BG management device, according to a difference between the blood glucose baseline and the blood glucose level of the patient after the specified time duration.
